(19) United States
(12) Patent Application Publication
(10) Pub. No.: US 2014/0200452 A1
(43) Pub. Date: Jul. 17, 2014

(54) USER INTERACTION BASED IMAGE SEGMENTATION APPARATUS AND METHOD

(71) Applicant: Samsung Electronics Co., Ltd., Suwon-si (KR)

(72) Inventors: Chu-Ho CHANG, Seoul (KR); Yeong-Kyeong SEONG, Yongin-si (KR); Ha-Young KIM, Hwaseong-si (KR); Kyoung-Gu WOO, Seoul (KR)

(73) Assignee: Samsung Electronics Co., Ltd., Suwon-si (KR)

(21) Appl. No.: 14/155,721

(22) Filed: Jan. 15, 2014

(30) Foreign Application Priority Data
Jan. 15, 2013 (KR) 10-2013-0004577

(51) Int. Cl. G06T 7/00; G06F 3/0484; A61B 8/08; G06F 3/0482

(52) U.S. Cl. CPC G06T 7/0081; G06T 7/0012; G06F 3/0482; G06F 3/04842; A61B 8/5215
USPC 600/437; 382/128; 715/810; 715/780; 600/407

(57) ABSTRACT

There is provided an image segmentation apparatus and related method for enhancing accuracy of image segmentation based on user interaction. The image segmentation apparatus including an interface configured to, in response to an image displayed on the interface, receive information about the image from a user and a segmenter configured to segment the contour of a region of interest (ROI) in the image based on the information.

### CROSS-REFERENCE TO RELATED APPLICATION

This application claims the benefit under 35 U.S.C. §119(a) of Korean Patent Application No. 10-2013-0004577, filed on Jan. 15, 2013, in the Korean Intellectual Property Office, the entire disclosure of which is incorporated herein by reference for all purposes.

### BACKGROUND

1. Field

The following description relates to an apparatus and method for enhancing accuracy of image segmentation based on user interaction.

2. Description of the Related Art

Generally, a contour of a region of interest (ROI), especially a mass or a lesion, such as a tumor, in a medical image is significant for a Computer-Aided Diagnosis (CAD) system to analyze the image and produce a result or a diagnosis. That is, if there is an accurate contour of an ROI, especially a lesion, it is possible to extract accurate features corresponding to the contour. Using the features derived from such an accurate contour, a lesion may be more accurately classified as benign or malignant, thereby enhancing accuracy of a diagnosis that specifies the nature of the lesion. Based on establishing the nature of lesion, a diagnosis improves the ability to treat the lesion.

However, there are limitations to providing a precise contour of an ROI in a general CAD system. Due to features that interfere with the quality of images, such as low resolution, low contrast, speckle noise and a blurred lesion boundary, such as of an ultrasound image, it is difficult for the CAD system to diagnose a lesion accurately or for a radiologist to analyze the ultrasound image so as to diagnose a lesion.

### SUMMARY

In one general aspect, there is provided an image segmentation apparatus including an interface configured to receive information about a displayed image comprising a region of interest (ROI), and a segmenter configured to segment a contour of the region of interest (ROI) in the image based on the received information.

The image segmentation apparatus may include that the received information includes approximate location information and the interface is configured to display a predetermined identification mark in the image at a location corresponding to the approximate location.

The interface may be further configured to displays a list of choices of information and to receive a user selection of a choice as received information.

The interface may be further configured to display a text input area and to receive user entry of text as received information.

The interface may be further configured to displays a list of recommended candidates and to allow the user to select received information from the list of recommended candidates.

The list of recommended candidates may be a list of lexicons which satisfy a predetermined requirement based on lexicons previously extracted with respect to the ROI.

The segmenter may be configured to segment the contour of the ROI by applying a level set method or a filtering method using the received information.

The segmenter may be configured to segment the contour by, in a level set equation, assigning a greater weighted value to a parameter corresponding to the received information.

The interface may be configured to displays the segmented contour in the image in an overlapping manner.

In another aspect, an image segmentation method includes receiving information via an interface about a displayed image comprising a region of interest (ROI), and segmenting a contour of the ROI in the image based on the received information.

The receiving information may include receiving approximate location information of the ROI and the method may further include displaying a predetermined identification mark at a corresponding location in the image.

The receiving information may include displaying a list of choices of information, and receiving a user selection of a choice as received information.

The receiving information may include displaying a text input area, and receiving a user entry of text as received information.

The receiving information may include displaying a list of recommended candidates, and allowing the user to select received information from the list of recommended candidates.

The list of recommended candidates may be a list of lexicons which satisfy a predetermined requirement based on lexicons previously extracted with respect to the ROI.

The segmenting the contour may include segmenting the contour by applying a level set method or a filtering method using the received information.

The segmenting the contour may include segmenting the contour by, in a level set equation, assigning a greater weighted value to a parameter corresponding to the received information.

The method may further include displaying the segmented contour in the image in an overlapping manner.

In another general aspect, there is provided a computer-aided diagnosis (CAD) apparatus, comprising an imager, configured to produce an image comprising a region of interest (ROI), an interface, configured to identify a candidate location of the ROI in the image, display the image to a user, including the candidate location, and receive information about the ROI from the user, a segmenter configured to segment a contour of the ROI based on the received information, and a computer-aided diagnoser, configured to diagnose the ROI based on the contour.

The image may be an ultrasound image of a patient.

The ROI may be a lesion.

The diagnosis may be an assessment of the severity of the lesion.

The information may be feature information described using a lexicon.

Other features and aspects will be apparent from the following detailed description, the drawings, and the claims.

### BRIEF DESCRIPTION OF THE DRAWINGS

The accompanying drawings, which are included to provide a further understanding of the invention and are incorporated in and constitute a part of this specification, illustrate embodiments of the invention, and together with the description serve to explain the principles of the invention.

FIG. 1 is a block diagram illustrating an image segmentation apparatus according to an exemplary embodiment.

FIGS. 2A to 2C are examples of an interface according to an exemplary embodiment.

FIG. 3 is a flowchart for illustrating an image segmentation method according to an exemplary embodiment.

### DETAILED DESCRIPTION

The following description is provided to assist the reader in gaining a comprehensive understanding of the methods, apparatuses, and/or systems described herein. Accordingly, various changes, modifications, and equivalents of the methods, apparatuses, and/or systems described herein will suggest themselves to those of ordinary skill in the art. Also, descriptions of well-known functions and constructions may be omitted for increased clarity and conciseness.

Throughout the drawings and the detailed description, the same reference numerals refer to the same elements. The drawings may not be to scale, and the relative size, proportions, and depiction of elements in the drawings may be exaggerated for clarity, illustration, and convenience.

The features described herein may be embodied in different forms, and are not to be construed as being limited to the examples described herein. Rather, the examples described herein have been provided so that this disclosure will be thorough and complete, and will convey the full scope of the disclosure to one of ordinary skill in the art.

Hereinafter, examples of an image segmentation apparatus based on user interaction and an image segmentation method based on user interaction will be described accompanied by drawings.

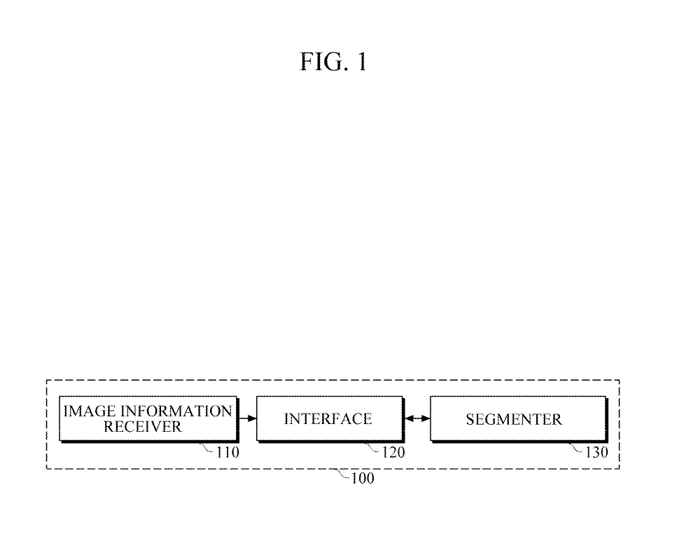

FIG. 1 is a block diagram illustrating an image segmentation apparatus according to an exemplary embodiment. An image segmentation apparatus 100 may be applied in a Computer Aided Diagnosis (CAD) system which analyzes an ultrasound image of a breast or other patient and provide a diagnosis thereof. For example, the goal of an example CAD system is to assess a mass in a breast and assess whether it is benign or malignant. Such an example CAD system is a system that receives a segmented image, and based on the segmentation of the image, uses use artificial intelligence techniques of various sorts to arrive at a diagnosis of a lesion or make treatment recommendation. In addition, the image segmentation apparatus 100 is able to segment a contour of a lesion, enhancing accuracy of a diagnosis. By segmenting a contour of the lesion, it is possible to more accurately assess its shape. Based on more accurate information about the shape of the lesion it is possible to enhance the performance of the CAD system. However, the above is merely an example. As another example, the image segmentation apparatus 100 may be applied in a general image processing system which needs to segment a contour of a region of interest (ROI). When imaging a ROI, the image quality will be greater if the image segmentation apparatus 100 is able to have better performance when segmenting a contour of a region of interest (ROI).

Hereinafter, embodiments are discussed that are related to an example of the image segmentation apparatus 100 that is applied in a CAD system to segment a contour of a lesion. While other embodiments are possible, the embodiment in the context of a CAD system is described for the sake of convenience of explanation.

Referring to FIG. 1, the image segmentation apparatus 100 may include an image information receiver 110, an interface 120 and a segmenter 130.

If an ultrasound image measuring device, such as a probe, which measures a medical image, scans a body part of a patient, the information gathered by the probe is processed and stored as an ultrasound image. After the ultrasound image is produced, the image information receiver 110 receives the ultrasound image of the body part.

The interface 120 provides a user-interaction-based interface to a user device and displays the received image on the interface. The user device may be a computer, a smart TV, a smart phone, a tablet PC and a laptop, which are connected to a display device, for example, a monitor, and provides an interface. In order to provide its capabilities, the interface 120 includes output and input components. For example, as discussed above, in embodiments the output components includes some form of display to present information to the user. In some embodiments, the display is a flat-panel monitor, such as an LED or LCD display. However, other embodiments may use another flat-panel technology, such as plasma, or a tube display technology, such as cathode ray tube (CRT) technology. In some embodiments, the interface outputs information to the user through other forms of output, such as audio outputs or printed output.

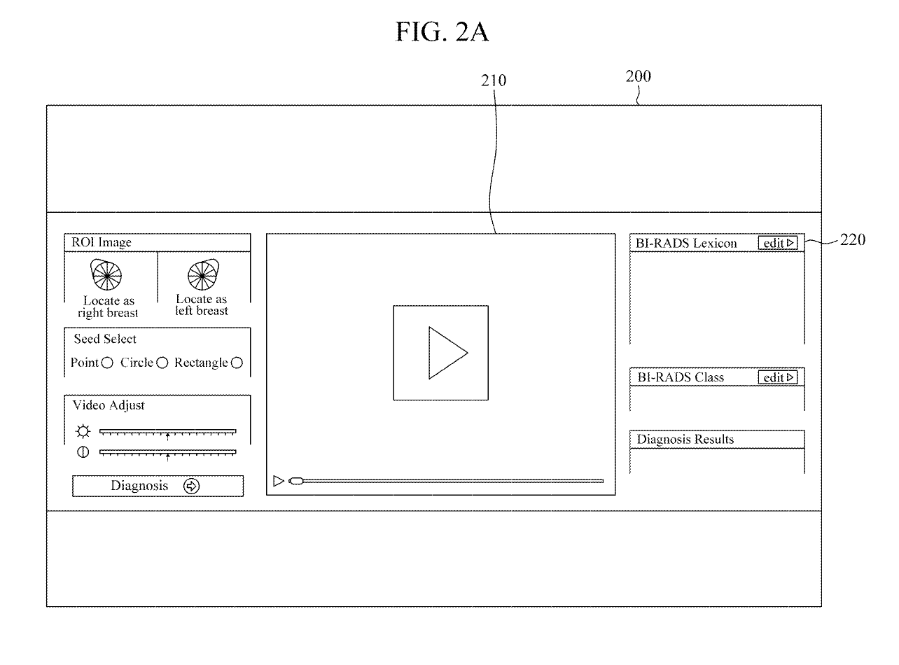

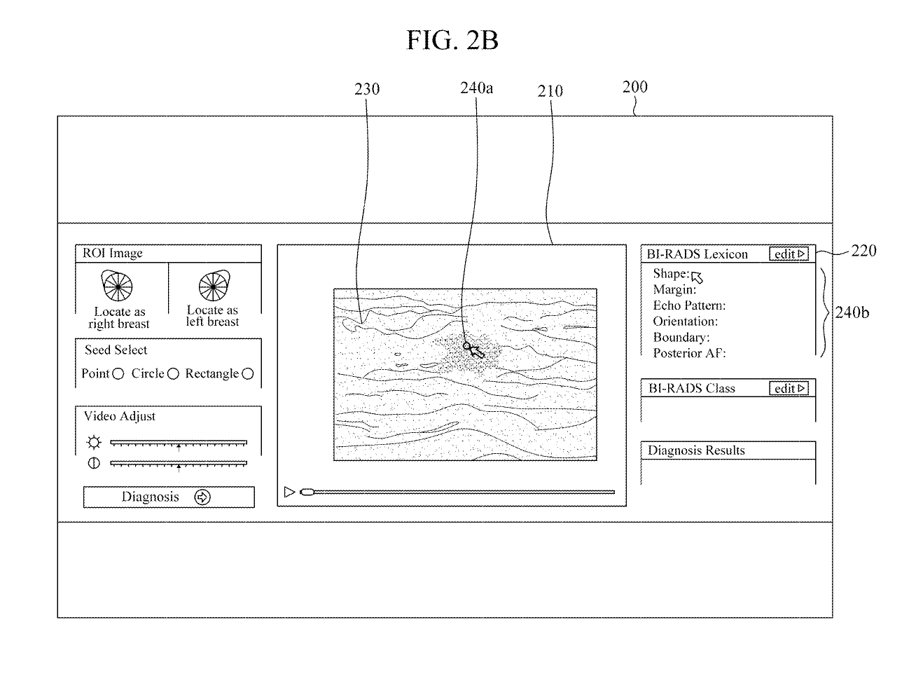

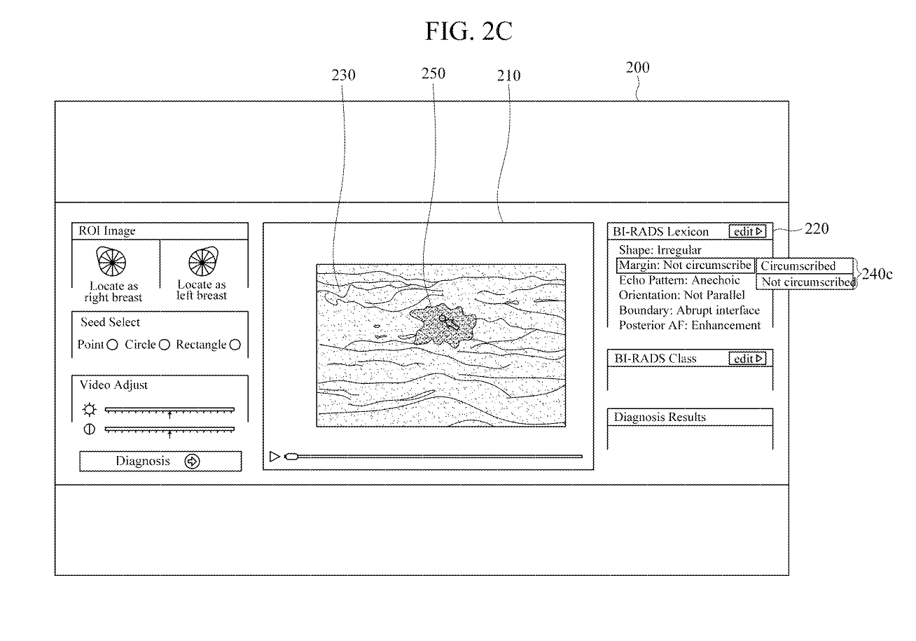

In addition, the interface 120 displays objects in various forms on the interface to allow a user to input additional information more easily. For example, the interface 120 provides a dropdown menu or a pop-up menu on which a user is able to select additional information, or may provide a text box in which a user is able to input text as additional information. The user is able to provide input to the interface 120 through use of various input devices and/or technologies. For example, the interface 120 receives input from a keyboard and/or a mouse. However, these are merely example input devices, and any other sort of input device such as a trackball, trackpad, microphone, touchscreen, etc. may be used by the user to provide input to the interface 120. More details about the interface and how it operates are provided in the discussion of FIGS. 2A-2C.

A user may input various types of additional information necessary for diagnosing an ROI, that is, a lesion, via the interface 120. For example, the additional information may be information that the user is aware of upon inspection of the ultrasound image, such as if the user is an experienced radiologist or otherwise perceives features of the ROI upon inspection of the ultrasound. Alternatively, the additional information may be information based on another image of the ROI, such as another ultrasound, or a different type of scanning technology such as a computerized tomography (CT) or magnetic resonance (MR) scan, or other knowledge about characteristics of the image, from any appropriate source.

For example, the user may input approximate location information of an ROI as additional information by identifying the ROI in an image displayed on an interface to be suspected. For example, the user may identify the location of the ROI, by drawing a boundary shape or identifying boundary points. In another example, the user may input feature information of an ROI using objects in various forms, which are provided on an interface, or other information which may affect the accuracy of a diagnosis of the ROI.

In this case, the feature information may be data based on a Breast Imaging-Reporting and Data System (BI-RADS) developed by the American College of Radiology (ACR). For example, BI-RADS may categorize lesions into different types. For example, the feature information may be a lexicon, such as descriptions of characteristics of lesions. For example, the characteristics may include a shape, a contour, an internal echo and a posterior echo of a lesion, and categories (for example, an irregular shape, a smooth contour and an unequal and rough internal echo) of the lexicon. The BI-RADS categories also include numerical assessment categories that identify the severity of a lesion.

If a user inputs additional information via an interface, the segmenter 130 segments a contour of an ROI based on the additional information. For example, the segmenter 130 may segment a contour of an ROI by applying received additional information in a level set method or a filtering method that processes graphics data to attempt to ascertain where a boundary region is located. Level sets use a numerical technique for tracking boundaries of shapes and solids. Many filtering methods exist, such as edge detection techniques that identify locations in images with dramatic color or brightness changes. Such locations, also known as discontinuities, may lie along a linear boundary and constitute an edge. Many methods, such as various ways of identifying discontinuities and the edges they are available to segment images. However, the above are merely examples, and other various methods in which additional information provided by a user is applied to segment a contour of an ROI are used in other embodiments.

The filtering method is a technique of displaying, as a result of segmentation of a contour, a type of contour that is selected to be most relevant contour. The selection of the most relevant contour is based on receiving additional information from among a plurality of proposed candidate contours generated by the CAD system. For example, if a user inputs additional information indicating that a shape is irregular, the segmenter 130 may display as a result of contour segmentation an irregular contour selected from among a plurality of candidate contours (for example, oral, round and irregular contours) generated by a CAD system. Thus, in this example, the CAD system and the user work together to segment a shape, in that the CAD generates a set of proposed alternative shapes, and the user is then able to discriminate between the proposals to help accept the best match.

The level set method is a known image segmentation technique, so a detailed description is not provided herein. In the level set method, the segmenter 130 segments a contour by, in a level set equation, assigning a greater weighted value to a parameter corresponding to input additional information.

For example, suppose that the greatest accuracy of segmentation is achieved where a value of A in the following Equation 1 is either a maximum or a minimum, that is, it is at an extreme value. In this case, if parameters α, β, γ and δ are calculated and used as part of the segmentation process so that A is at such an extreme value, accuracy of segmentation may be enhanced. In certain embodiments, the parameters α, β, γ and δ are calculated using a regression analysis technique or a neural network technique. These techniques provide estimated values of the parameters that are designed to produce extreme values and thereby produce the best segmentation results.

A=α×I_{global}_{_{—}}_{region}+β×I_{local}_{_{—}}_{region}+γ×C_{edge}+δ×Cₛₘₒₒₜₕₙₑₛₛ+ . . . Equation 1

In the above Equation 1, the values related to I and C denote values relating to an image and a contour, respectively. Equation 1 includes an ellipsis at its end, indicating that other terms may be summed to produce A, if they provide useful information about segmentation results.

For the terms provided, I_{global}_{_{—}}_{region} denotes energy of an entire image in image information; I_{local}_{_{—}}_{region} indicates energy of an area surrounding a contour of the image in image information; C_{edge} means an edge component of the contour in contour information; and Cₛₘₒₒₜₕₙₑₛₛ represents a level of smoothness in a curve in contour information. In this context, energy may be a measure of the information contained in an image, related to its entropy.

In the level set equation, parameters α, β, γ and δ are calculated using additional information input by a user by employing a regression analysis technique or a neural network technique. However, as noted, it is possible to use other types of analysis to find values of these parameters. That is, the level set equation improves accuracy of segmentation of a contour by giving a greater weighted value to a parameter corresponding to additional information input by a user. The accuracy is improved because if the user's input is given more weight, the user's input potentially corrects errors that otherwise would have interfered with segmentation results.

For example, if a user inputs additional information indicating that a shape is irregular, a more irregular contour may be generated by assigning a greater weighted value to parameter 6 relating to smoothness of a contour curve while adjusting weighted values assigned to other parameters. If the weights are adjusted based on knowledge derived from the user, it helps optimize the parameters because the knowledge from the user helps to determine which parameters should be emphasized when determining the contour. Pieces of additional information input by a user may correspond to various parameters concurrently, and, every piece of the additional information may be reflected in a weighted value of each parameter. At this point, information about a parameter corresponding to additional information input by a user or information on a weighted value to be given to each of the parameters may be set in advance by the user. In addition, if additional information affects other additional information, different weighted values may set to be assigned to the other parameters according to how much the additional information affects each parameter. For example, different characterizations of smoothness or image energy may cause the parameters to change, and may change different parameters at the same time. As discussed, some embodiments use predefined impacts of various user inputs on the weighting. However, some alternative embodiments provide users with the ability to control what effect and how much of an effect corresponds with their input.

FIGS. 2A to 2C are examples of an interface according to an exemplary embodiment. Referring to FIG. 1 and FIGS. 2A to 2C, an interface provided in an image segmentation apparatus 100 and a method for inputting additional information by a user using the interface will be described.

As illustrated in FIG. 2A, an interface 120 provides an interface 200 to a user device. The interface 200 is an example of an interface which provides a user with a medical image scanned in a CAD system and a diagnosis thereof. For example, the interface 200 shown in FIG. 2A is a graphical user interface (GUI) that includes windows that display information to the user and receive inputs from the user in order to gather input information, such as information that characterizes a lesion. However, the interface 200 presented in FIG. 2A is merely an example. Various other embodiments are generated or modified to be in various forms so as to provide a user with an interface optimized for user convenience in the context of an applied segmentation system.

As illustrated in FIG. 2A, the interface 200 may include a first area 210, in which a received image is displayed, and a second area 220 to which a user may input additional information. The interface 200 may display in the first area 210 an image received by an image information receiver 110, and display in the second area 220 various graphic objects and controls to allow a user to input additional information so as to segment a contour in the image displayed in the first area 210 based on the additional information.

Referring to FIG. 2B, a medical image 230 is displayed in the first area 210 of the interface 200. A user may input appropriate location information as additional information on a region of interest (ROI) suspected of being a lesion in the displayed medical image 230. In one example, the user designates an ROI by selecting a location of the ROI using an input means, such as a mouse, a finger, and/or a stylus pen, or by distinguishing the ROI to be in a form of circle, oval and square. Alternatively, the user designates points that define a polygon or curve bounding the location of the ROI. In response to the user's inputting the appropriate location information of the ROI, the interface 120 displays a predetermined identification mark 240a at a corresponding location in the image. For example, the predetermined identification mark 240a in FIG. 2B is an identified point located roughly in the center of the lesion. At this point, in some embodiments the identification mark 240a is displayed with various colors so as to be easily recognized by the user. For example, if the displayed medical image 230 is shown in grayscale or flesh tones, a bright color such as blue, green, or red is used to color the identification mark 240a so that it is easily recognizable. However, these are only example colors and other ways of making the identification mark 240a visible are available to other embodiments.

Referring to FIGS. 2B and 2C, the interface 120 may display in the second area 220 a list 240b of additional information lexicons and a list 240c of additional information categories corresponding to the lexicons that work together to allow a user to select desired additional information from the list 240b or from the list 240c. For example, the additional information categories include different types of information about the lesion that a user wishes to specify. For example, FIG. 2B illustrates that these categories include shape, margin, echo pattern, orientation, boundary, posterior AF, and so on. These are illustrative examples of categories from the BI-RADS lexicon. Different embodiments include different lexicons in the list 240b lead to lists 240c of varying additional information categories, in that optionally, additional categories are included, and optionally, not all of these example categories are included. As discussed, a list of additional information may be a list of BI-RADS lexicons. However, the above is merely an example type of additional information, and a list of additional information may include any information which may affect contour segmentation.

For example, as illustrated in FIG. 2B, the interface 120 may display a list 240b of lexicons in the second area 220. If a user selects a lexicon (for example, margin) from the list 240b, a list of categories 240c (for example, circumscribed and not circumscribed) of the selected lexicon may be displayed in a pop-up window, as illustrated in FIG. 2C. However, the is above is merely an example, a list of categories of a lexicon may be displayed in any form. For example, a list of categories of a selected lexicon may be displayed in a different area, for example, a bottom part, of the interface 200. In general, the interface 200 receives information from a user by selecting a lexicon, and then selecting an information category included in the lexicon.

In addition, in certain embodiments the interface 120 displays in the second area 220 a text input area (not shown), such as a text input to allow a user to input text in the text input area as additional information. This use of text as a type of input provides an alternative way to gather information from the user, instead of relying on the drop-down approach of selecting lexicons and corresponding categories with predefined choices.

Accordingly, a user may input text as additional information, instead of selecting additional information on a displayed list of additional information. Alternatively, a user may select partial additional information on a displayed list of additional information while inputting additional information, which is provided on the displayed list of additional information, in the form of text. For example, a user may characterize their textual comments as belonging to a certain category of information, but still enter the information as text. For example, the textual comments might include information that is classified as “medical history” and includes additional diagnostic notes from a user.

The interface 120 may receive additional information input by voice from a user through a voice input device which is installed in an image segmentation apparatus. For example, instead of entering text, a user may dictate comments for recognition by a speech recognizer. Alternatively, the user uses a speech recognizer to choose a lexicon and a category within the lexicon, as discussed above.

In another general aspect, the interface 120 provides a list of recommended candidates to allow a user to select desired additional information to be input in the second area 220 from the list of recommended candidates. By providing recommended candidates to the user, the interface 120 helps the user by identifying which information from the user will be most helpful for improving system performance. At this point, the list of recommended candidates may include a list of lexicons which satisfy a predetermined requirement among previously extracted lexicons. That is, embodiments may track information that was previously received from the user and determine which information was the most helpful. Based on this tracked information, embodiments are able to determine categories to present to the user. In an example, the interface 120 determines that information about shape and boundary are especially helpful information when trying to improve recognition of a lesion. Alternatively, types of commentary may be flagged as being especially helpful as well. For example, comments on family history may be especially helpful. A list of recommended candidates may be generated by a CAD diagnosis system and transmitted to the image information receiver 110.

In some embodiments, when choosing a lexicon that is suitable for embodiments, the lexicon is chosen to satisfy a predetermined requirement. For example, the lexicon is chosen to be a lexicon whose level of affecting a result of a diagnosis in a CAD diagnosis system exceeds a predetermined threshold. Such a choice may help guarantee that using the lexicon has a useful impact on the CAD diagnosis process and is advantageous. For example, a predetermined threshold may be set so as to provide a list of recommended candidates indicating lexicons whose level of affecting a result of a diagnosis in a CAD diagnosis system is uncertain, and the list of recommended candidates may be determined using decision tree methods and the like to choose from among the recommended candidates. In decision tree methods, decisions are progressively made that provide strategies that incorporate series of decisions.

As such, a user is provided with lexicons, whose level of affecting a result of a diagnosis in a CAD diagnosis system is uncertain, on a list of recommended candidates. If the user inputs additional information on a recommended candidate which is considered most significant in a result of a diagnosis out of all the recommended candidates, a diagnosis is performed using the input additional information, thereby enhancing accuracy of the diagnosis.

If a user inputs additional information by selecting the additional information on a list 240c of additional information or if a user inputs additional information by using one of text, voice input or a list of recommended candidates, the segmenter 130 segments a contour of an ROI using the input additional information. For example, a user provides that a ROI has a margin that is “circumscribed” or “not circumscribed.” Based on this information about the ROI, the segmenter 130 may model the ROI in different ways and use the modeling to improve segmentation performance. As such, if the contour of the ROI is segmented by the segmenter 130, the interface 20 may displays a contour 250 located in an image, displayed in the first area 210, based on information on the contour 250 in an overlapping manner.

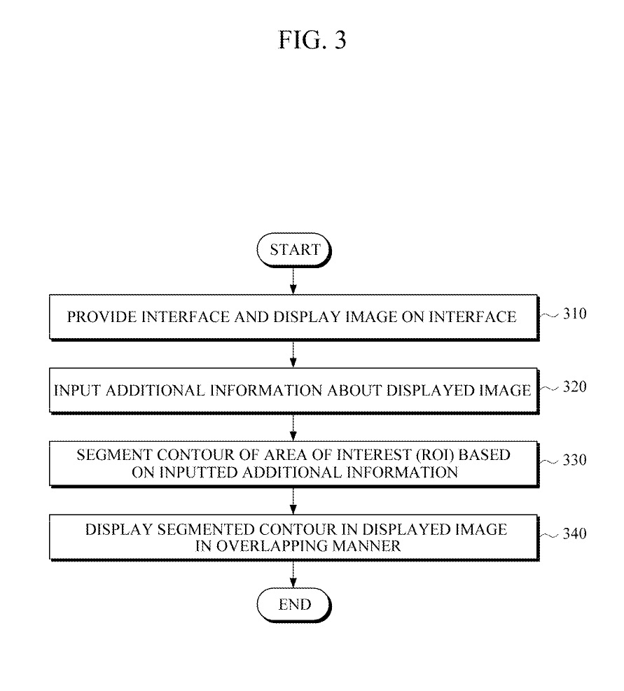

FIG. 3 is a flowchart illustrating an image segmentation method according to an exemplary embodiment. That is, FIG. 3 shows an example in which the image segmentation apparatus 100 shown in FIG. 1 segments a contour of an image. As the image segmentation method was already described in detail with reference to FIGS. 1 and 2, the image segmentation method will be explained briefly in the following.

The image segmentation apparatus 100 provides an interface to a user device, and displays an image which is input to the interface in 310. For example, the input image is a medical image scanned in real time by an ultrasonic measuring device, such as a probe.

Next, if the user inputs additional information through the interface, the image segmentation apparatus 100 receives the additional information in 320. In an example, the user inputs approximate location information of an ROI, which is suspected of being a lesion in the image displayed on the interface, as additional information. At this point, the user inputs the approximate location information of an ROI using various methods as described above. In an embodiment, the image segmentation apparatus 100 displays various identification marks on the interface in response to user inputs. In an example, the identification marks identify a center of the ROI.

In addition, in some embodiments the user inputs further additional information necessary to segment the lesion-suspected ROI more accurately. In examples, the additional information includes lexicons and categories of the lexicons. For example, the user may input the additional information by entering text as additional information or selecting additional information from a list of choices of additional information, which is displayed on the interface.

At this point, in the event that a list of recommended candidates, which was previously generated by a CAD diagnosis system, is input along with a corresponding medical image, the image segmentation apparatus 100 displays the list of recommended candidates on the interface, thereby allowing the user to input further accurate additional information on the ROI. Alternatively, in the event that a medical image is displayed, if the user inputs approximate location information of an ROI suspected of being a lesion in the medical image and then requests a list of recommended candidates relevant to the ROI, the image segmentation apparatus 100 may request the list of recommended candidates from a CAD diagnosis system.

Next, the segmentation apparatus 100 segments a contour of the ROI based on the input additional information in 330. For example, as described above, it is possible to segment a contour of an ROI using a level set method or a filtering method. In the case of the level set method, a weighted value corresponding to the inputted additional information may be set to be greater than that of other additional information, thereby enabling more accurate segmentation of the contour. Similarly, a filtering method may also take into account the additional information when performing the segmentation operation.

If the contour of the ROI is segmented accurately, the image segmentation apparatus 100 displays the contour at a location corresponding to the medical image in the interface in an overlapping manner in 340.

In the embodiments described above, by segmenting a contour of a ROI based on additional information input by a user, it is possible to more accurately segment the contour and, in turn, achieve a more precise result of a diagnosis than that of diagnosing a lesion by a CAD diagnosis system only using medical images. While initial estimates of the location of the lesion are based on automated results produced by the CAD diagnosis system, the user is able to use various forms of input to improve the accuracy of the contour of the ROI. A more accurate contour provides the CAD system with information about the ROI that makes it easier to diagnose, as having more information, and more accurate information about the ROI will make it more likely that the CAD system can provide diagnoses that are correct and accurate.

Meanwhile, the exemplary embodiments of the present invention may be realized using computer-readable codes in a computer-readable recording medium. The computer-readable recording medium includes all types of recording devices which stores computer-system readable data.

Examples of the computer-readable recording medium includes a Read Only Memory (ROM), a Random Access Memory (RAM), a CD-ROM, a magnetic tape, a floppy disk and an optical data storage device, and the computer readable recording medium may be realized in a carrier wave form (for example, transition via the Internet). In addition, the computer-readable recording medium is distributed in a computer system connected via a network so that computer-readable codes are stored and executed in a distributed manner. In addition, functional programs, codes and code segments used to embody the present invention may be easily anticipated by programmers in the technical field of the present invention.

Program instructions to perform a method described herein, or one or more operations thereof, may be recorded, stored, or fixed in one or more computer-readable storage media. The program instructions may be implemented by a computer. For example, the computer may cause a processor to execute the program instructions. The media may include, alone or in combination with the program instructions, data files, data structures, and the like. Examples of computer-readable storage media include magnetic media, such as hard disks, floppy disks, and magnetic tape; optical media such as CD ROM disks and DVDs; magneto-optical media, such as optical disks; and hardware devices that are specially configured to store and perform program instructions, such as read-only memory (ROM), random access memory (RAM), flash memory, and the like. Examples of program instructions include machine code, such as produced by a compiler, and files containing higher level code that may be executed by the computer using an interpreter. The program instructions, that is, software, may be distributed over network coupled computer systems so that the software is stored and executed in a distributed fashion. For example, the software and data may be stored by one or more computer readable storage mediums. Also, functional programs, codes, and code segments for accomplishing the example embodiments disclosed herein can be easily construed by programmers skilled in the art to which the embodiments pertain based on and using the flow diagrams and block diagrams of the figures and their corresponding descriptions as provided herein. Also, the described unit to perform an operation or a method may be hardware, software, or some combination of hardware and software. For example, the unit may be a software package running on a computer or the computer on which that software is running.

As a non-exhaustive illustration only, a terminal/device/unit described herein may refer to mobile devices such as a cellular phone, a personal digital assistant (PDA), a digital camera, a portable game console, and an MP3 player, a portable/personal multimedia player (PMP), a handheld e-book, a portable laptop PC, a global positioning system (GPS) navigation, a tablet, a sensor, and devices such as a desktop PC, a high definition television (HDTV), an optical disc player, a setup box, a home appliance, and the like that are capable of wireless communication or network communication consistent with that which is disclosed herein.

A computing system or a computer may include a microprocessor that is electrically connected with a bus, a user interface, and a memory controller. It may further include a flash memory device. The flash memory device may store N-bit data via the memory controller. The N-bit data is processed or will be processed by the microprocessor and N may be 1 or an integer greater than 1. Where the computing system or computer is a mobile apparatus, a battery may be additionally provided to supply operation voltage of the computing system or computer. It will be apparent to those of ordinary skill in the art that the computing system or computer may further include an application chipset, a camera image processor (CIS), a mobile Dynamic Random Access Memory (DRAM), and the like. The memory controller and the flash memory device may constitute a solid state drive/disk (SSD) that uses a non-volatile memory to store data.

A number of examples have been described above. Nevertheless, it will be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

A number of examples have been described above. Nevertheless, it should be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

What is claimed is:

1. An image segmentation apparatus comprising:
an interface configured to receive information about a displayed image comprising a region of interest (ROI); and
a segmenter configured to segment a contour of the region of interest (ROI) in the image based on the received information.

2. The image segmentation apparatus of claim 1, wherein the received information comprises approximate location information and the interface is configured to display a predetermined identification mark in the image at a location corresponding to the approximate location.

3. The image segmentation apparatus of claim 1, wherein the interface is further configured to displays a list of choices of information and to receive a user selection of a choice as received information.

4. The image segmentation apparatus of claim 1, wherein the interface is further configured to display a text input area and to receive user entry of text as received information.

5. The image segmentation apparatus of claim 1, wherein the interface is further configured to displays a list of recommended candidates and to allow the user to select received information from the list of recommended candidates.

6. The image segmentation apparatus of claim 5, wherein the list of recommended candidates is a list of lexicons which satisfy a predetermined requirement based on lexicons previously extracted with respect to the ROI.

7. The image segmentation apparatus of claim 1, wherein the segmenter is configured to segment the contour of the ROI by applying a level set method or a filtering method using the received information.

8. The image segmentation apparatus of claim 7, wherein the segmenter is configured to segment the contour by, in a level set equation, assigning a greater weighted value to a parameter corresponding to the received information.

9. The image segmentation apparatus of claim 1, wherein, the interface is configured to displays the segmented contour in the image in an overlapping manner.

10. An image segmentation method comprising:
receiving information via an interface about a displayed image comprising a region of interest (ROI); and
segmenting a contour of the ROI in the image based on the received information.

11. The image segmentation method of claim 10, wherein the receiving information comprises receiving approximate location information of the ROI and the method further comprises:
displaying a predetermined identification mark at a corresponding location in the image.

12. The image segmentation method of claim 10, wherein the receiving information comprises:
displaying a list of choices of information; and
receiving a user selection of a choice as received information.

13. The image segmentation method of claim 10, wherein the receiving information comprises:
displaying a text input area; and receiving a user entry of text as received information.

14. The image segmentation method of claim 10, wherein the receiving information comprises:
displaying a list of recommended candidates; and
allowing the user to select received information from the list of recommended candidates.

15. The image segmentation method of claim 14, wherein the list of recommended candidates is a list of lexicons which satisfy a predetermined requirement based on lexicons previously extracted with respect to the ROI.

16. The image segmentation method of claim 10, wherein the segmenting the contour comprises segmenting the contour by applying a level set method or a filtering method using the received information.

17. The image segmentation method of claim 16, wherein the segmenting the contour comprises segmenting the contour by, in a level set equation, assigning a greater weighted value to a parameter corresponding to the received information.

18. The image segmentation method of claim 10, further comprising:
displaying the segmented contour in the image in an overlapping manner.

19. A computer-aided diagnosis (CAD) apparatus, comprising:
an imager, configured to produce an image comprising a region of interest (ROI);
an interface, configured to:
identify a candidate location of the ROI in the image;
display the image to a user, including the candidate location; and
receive information about the ROI from the user;
a segmenter configured to segment a contour of the ROI based on the received information; and
a computer-aided diagnoser, configured to diagnose the ROI based on the contour.

20. The apparatus of claim 19, wherein the image is an ultrasound image of a patient.

21. The apparatus of claim 19, wherein the ROI is a lesion.

22. The apparatus of claim 21, wherein the diagnosis is an assessment of the severity of the lesion.

23. The apparatus of claim 19, wherein the information is feature information described using a lexicon.
